(19) United States
(12) Patent Application Publication
(10) Pub. No.: US 2008/0227166 A1
(43) Pub. Date: Sep. 18, 2008

(54) Fermentation Processes

(75) Inventors: Eric Allain, Boone, NC (US); Kevin S. Wenger, Wake Forest, NC (US); Henrik Bisgard-Frantzen, Bagsvaerd (DK); Lori Henderson, Raleigh, NC (US); Carmen Costable, North Chili, NY (US)

Correspondence Address: NOVOZYMES NORTH AMERICA, INC., 500 FIFTH AVENUE, SUITE 1600, NEW YORK, NY, 10110, US

(73) Assignees: Novozymes A/S, Bagsvaerd (DK); Novozymes North America, Inc., Franklinton, NC (US)

(21) Appl. No.: 10/586,097

(22) PCT Filed: Jan. 14, 2005
(86) PCT No.: PCT/US2005/001170

§ 371 Date: Nov. 2, 2006

Related U.S. Application Data
(60) Provisional application No. 60/537,071, filed on Jan. 16, 2004.

(51) Int. Cl. C12P 7/06

(52) U.S. Cl. 435/161

(57) ABSTRACT

The present invention provides an improved process for producing a fermentation product in a fermentation medium, including ethanol, which process include a fermentation step, comprising subjecting the fermentation medium to at least one surfactant and at least one carbohydrate-source generating enzyme.

### BACKGROUND OF THE INVENTION

1. Field of the Invention

The present invention relates to enzymatic processes for producing fermentation products, including processes for producing ethanol.

2. Detailed Description of the Related Art

Fermentation processes are used for making a vast number of commercial products, including alcohols (e.g., ethanol, methanol, butanol 1,3-propanediol); organic acids (e.g., citric acid, acetic acid, itaconic acid, lactic acid, gluconic acid, gluconate, lactic acid, succinic acid, 2,5-diketo-D-gluconic acid); ketones (e.g., acetone); amino acids (e.g., glutamic acid); gases (e.g., H₂ and CO₂), and more complex compounds, including, for example, antibiotics (e.g., penicillin and tetracycline); enzymes; vitamins (e.g., riboflavin, B₁₂, beta-carotene); hormones, and other compounds which are difficult to produce synthetically. Fermentation processes are also commonly used in the consumable alcohol (e.g., beer and wine), dairy (e.g., in the production of yoghurt and cheese), leather, and tobacco industries.

There is a need for further improvement of fermentation processes and for improved processes which include a fermentation step.

### SUMMARY OF THE INVENTION

In the first aspect the present invention provides an improved fermentation processes for producing a fermentation product in a fermentation medium comprising subjecting a fermentation medium to at least one surfactant and at least one carbohydrate-source generating enzyme.

In a second aspect the invention relates to an improved process for producing ethanol comprising the steps of:

(a) milling whole grains;

(b) liquefying the product of step (a);

(c) saccharifying the liquefied material obtained in step (b);

(d) fermenting the saccharified material using a fermenting microorganism, wherein the fermentation process further comprises contacting the fermentation media with at least one surfactant, at least one carbohydrate-source generating enzyme.

### BRIEF DESCRIPTION OF THE DRAWING

FIG. 1 shows the result of SSF in 16 mL tubes using glucoamylase, cellulase and/or SOFTANOL® 90 surfactant.

FIG. 2 shows the result of SSF in 16 mL tubes using glucoamylase, cellulase and/or TRITON® X100 surfactant.

FIG. 3 shows the result of SSF in 16 mL tubes using glucoamylase, cellulase and/or BEROL® 087 surfactant.

### DESCRIPTION OF THE INVENTION

The present invention provides an improved fermentation processes for producing a fermentation product. The present invention also provides improved processes for producing ethanol using one or more of the processes described herein. According to the invention a higher fermentation yield may be obtained.

In the first aspect the invention relates to a process for producing a fermentation product in a fermentation medium which process includes a fermentation step, comprising subjecting the fermentation medium to at least one surfactant and at least one carbohydrate-source generating enzyme.

### Surfactants

The surfactant may according to the invention be any surfactant. Preferred surfactants are non-ionic surfactants. Preferred surfactants include alcohol ethoxylates, alcohol propoxylates, alcohol ethoxylate-propoxylates, alcohol, ethoylates, oxa alcohol, ethoylates, copolymeric carboxylate dispersant, and polyacrylic dispensant. Most preferred surfactants are alcohol ethoxylates. In a preferred embodiment the surfactant is a fatty alcohol ethoxylate, a C₁₂₋₁₄ secondary alcohol ethoxylate, or a C₁₂₋₁₄ secondary alcohol ethoxylate reacted with ethylene oxide, a mixture of C₁₁₋₁₅ secondary alcohols reacted with ethylene oxide. The surfactant may also be an octylphenol ethoxylate or a C₁₁₋₁₅H₂₃₋₃₁O(CH₂CH₂O)ₓH.

Examples of commercially available surfactant products include BEROL™ 087 from Akzo Nobel, SOFTANOL™ 50, SOFTANOL™ 90 from Honeywell & Stein, TRINTON™ X-100, TERGITOL™ 15-S-5, TERGITOL™ 15-S-9 from Union Carbide.

### Carbohydrate-Source Generating Enzyme

The term “carbohydrate-source generating enzyme” includes glucoamylases (being a glucose generators), and beta-amylases and maltogenic amylases (being maltose generators). A carbohydrate-source generating enzyme is capable of providing energy to the fermenting microorganism(s) used in the process of the invention and/or may be converting directly or indirectly to the desired fermentation product, preferably ethanol. The carbohydrate-source generating enzyme may be mixtures of enzymes falling within the definition. Especially contemplated mixtures are mixtures of at least a glucoamylase and an alpha-amylase, especially an acid amylase, even more preferred an acid fungal alpha-amylase. The ratio between acidic fungal alpha-amylase activity (AFAU) per glucoamylase activity (AGU) (AFAU per AGU) may in an embodiment of the invention be at least 0.1, in particular at least 0.16, such as in the range from 0.12 to 0.50.

Examples of contemplated glucoamylases, alpha-amylases and beta-amylases are set forth in the sections below.

It is to be understood that the enzymes used according to the invention should be added in effective amounts.

### Glucoamylase

A glucoamylase used according to the invention may be derived from any suitable source, e.g., derived from a microorganism or a plant. Preferred glucoamylases are of fungal or bacterial origin, selected from the group consisting of Aspergillus glucoamylases, in particular A. niger G1 or G2 glucoamylase (Boel et al. (1984), EMBO J. 3 (5), p. 1097-1102), or variants thereof, such as disclosed in WO 92/00381, WO 00/04136 add WO 01/04273 (from Novozymes, Denmark); the A. awamori glucoamylase (WO 84/02921), A. oryzae (Agric. Biol. Chem. (1991), 55 (4), p. 941-949), or variants or fragments thereof.

Other Aspergillus glucoamylase variants include variants to enhance the thermal stability: G137A and G139A (Chen et al. (1996), Prot. Eng. 9, 499-505); D257E and D293E/Q (Chen et al. (1995), Prot. Engng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Engng. 10, 1199-1204. Other glucoamylases include Corticium rolfsii glucoamylase (U.S. Pat. No. 4,727,046) also referred to as Athelia rolfsii, Talaromyces glucoamylases, in particular, derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215). Bacterial glucoamylases contemplated include glucoamylases from the genus Clostridium, in particular C. thermoamylolyticum (EP 135,138), and C. thermohydrosulfuricum (WO 86/01831).

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYM™ B4U and AMG™ E (from Novozymes A/S); OPTIDEX™ 300 (from Genencor Int.); AMIGASE™ and AMIGASE™ PLUS (from DSM); G-ZYME™ G900, G-ZYME™ and G990 ZR (from Genencor Int.).

Glucoamylases may in an embodiment be added in an amount of 0.02-20 AGU/g DS, preferably 0.1-10 AGU/g DS, such as 2 AGU/g DS.

### Amylase

According to the invention preferred alpha-amylases are of fungal or bacterial origin.

More preferably, the alpha-amylase is a Bacillus alpha-amylase, such as, derived from a strain of B. licheniformis, B. amyloliquefaciens, and B. stearothermophilus. Other alpha-amylases include alpha-amylase derived from a strain of the Bacillus sp. NCIB 12289, NCIB 125121, NCIB 12513 or DSM 9375, all of which are described in detail in WO 95/26397, and the alpha-amylase described by Tsukamoto et al., Biochemical and Biophysical Research Communications, 151 (1988), pp. 25-31. Other alpha-amylase variants and hybrids are described in WO 96/23874, WO 97/41213, and WO 99/19467. Other alpha-amylase includes alpha-amylases derived from a strain of Aspergillus, such as, Aspergillus oayzae and Aspergillus niger alpha-amylases. In a preferred embodiment, the alpha-amylase is an acid alpha-amylase. In a more preferred embodiment the acid alpha-amylase is an acid fungal alpha-amylase or an acid bacterial alpha-amylase. More preferably, the acid alpha-amylase is an acid fungal alpha-amylase derived from the genus Aspergillus. A commercially available acid fungal amylase is SP288 (available from Novozymes A/S, Denmark).

In a preferred embodiment, the alpha-amylase is an acid alpha-amylase. The term “acid alpha-amylase” means an alpha-amylase (E.C. 3.2.1.1) which added in an effective amount has activity at a pH in the range of 3.0 to 7.0, preferably from 3.5 to 6.0, or more preferably from 4.0-5.0.

A preferred acid fungal alpha-amylase is an alpha-amylase which exhibits a high homology (identity), i.e. more than 50%, 55%, 60%, 65%, 70%, 75%, 80%, 85% 90%, or even at least 95%, 97%, 99% homology (identity) to the amino acid sequence shown in SEQ ID NO: 10 in WO 96/23874. When used as a maltose generating enzyme, fungal alpha-amylases may be added in an amount of 0.001-1.0 AFAU/g DS, preferably from 0.002-0.5 AFAU/g DS, preferably 0.02-0.1 AFAU/g DS.

Preferably the alpha-amylase is an acid alpha-amylase, preferably an add fungal alpha-amylase, more preferably from the genus Aspergillus, even more preferably of the species Aspergillus niger. In a preferred embodiment the acid fungal alpha-amylase is the one from A. niger disclosed as “AMYA_ASPNG” in the Swiss-prot/TeEMBL database under the primary accession no. P56271. Also variant of said acid fungal amylase having at least 70% identity, such as at least 80% or even at least 90%, 95%, 97% or 99% identity thereto is contemplated.

A preferred acid alpha-amylase for use in the present invention may be derived from a strain of B. licheniformis, B. amyloliquefaciens, and B. stearothermophilus.

Preferred commercial compositions comprising alpha-amylase include MYCOLASE from DSM (Gist Brdchades), BAN™, TERMAMYL™ SC, FUNGAMYL™, LIQUOZYME™ X and SAN™ SUPER, SAN™ EXTRA L (Novozymes A/S) and CLARASE™ L-40,000, DEX-LO™, SPEYME FRED, SPEZYME™ M, and SPEZYME™ DELTA AA (Genencor Int.), and the acid fungal alpha-amylase sold under the trade name SP 288 (available from Novozymes A/S, Denmark).

The amylase may also be a maltogenic alpha-amylase. A “maltogenic alpha-amylase” (glucan 1,4-alpha-maltohydrolase, E.C. 3.2.1.133) is able to hydrolyze amylose and amylopectin to maltose in the alpha-configuration. A maltogenic alpha-amylase from Bacillus stearothermophilus strain NCIB 11837 is commercially available from Novozymes A/S. Maltogenic alpha-amylases are described in U.S. Pat. Nos. 4,598,048, 4,604,355 and 6,162,628, which are hereby incorporated by reference. Preferably, the maltogenic alpha-amylase is used in a raw starch hydrolysis process, as described, e.g., in WO 95/10627, which is hereby incorporated by reference.

The alpha-amylase may be added in amounts as are well-known in the art. When measured in AAU units the acid alpha-amylase activity is preferably present in an amount of 5-50,0000 MU/kg of DS, in an amount of 500-50,000 MU/kg of DS, or more preferably in an amount of 100-10,000 MU/kg of DS, such as 500-1,000 MU/kg DS. Fungal acid alpha-amylase are preferably added in an amount of 10-10,000 AFAU/kg of DS, in an amount of 500-2,500 AFAU/kg of DS, or more preferably in an amount of 100-1,000 AFAU/kg of DS, such as approximately 500 AFAU/kg DS.

### Beta-amylase

At least according to the invention the a beta-amylase (E.C 3.2.1.2) is the name traditionally given to exo-acting maltogenic amylases, which catalyze the hydrolysis of 1,4-alpha-glucosidic linkages in amylose, amylopectin and related glucose polymers. Maltose units are successively removed from the non-reducing chain ends in a step-wise manner until the molecule is degraded or, in the case of amylopectin, until a branch point is reached. The maltose released has the beta anomeric configuration, hence the name beta-amylase.

Beta-amylases have been isolated from various plants and microorganisms (W. M. Fogarty and C. T. Kelly, Progress in Industrial Microbiology, vol. 15, pp. 112-115, 1979). These beta-amylases are characterized by having optimum temperatures in the range from 40° C. to 65° C. and optimum pH in the range from 4.5 to 7. A commercially available beta-amylase from barley is SPEZYME™ BBA 1500 from Genencor Int., USA.

### Maltogenic Amylase

Maltogenic amylases (glucan 1,4-alpha-maltohydrolase, E.C. 3.2.1.133) are able to hydrolyse amylose and amylopectin to maltose in the alpha-configuration. Furthermore, a maltogenic amylase is able to hydrolyse maltotriose as well as cyclodextrin. A specifically contemplated maltogenic amylase includes the one disclosed in EP patent no. 120,693 derived from Bacillus stearothermophilus C599. A commercially available maltogenic amylase is MALTOGENASE™ from Novozymes A/S.

In an embodiment of the present invention at least one surfactant and carbohydrate-source generating enzyme is applied to the fermentation medium before or during fermentation. In a preferred embodiment, the invention comprises contacting the fermentation medium with at least one surfactant and at least one carbohydrate-source generating enzyme. In another embodiment at least one surfactant and at least one carbohydrate-source generating enzyme treatment is performed directly on the fermentation media. In a preferred embodiment at least one surfactant and at least one carbohydrate-source generating enzyme is added directly to the fermentation medium. In an embodiment at least one surfactant and at least one carbohydrate-source generating enzyme is added before or during fermentation process. At least one surfactant and at least one carbohydrate-source generating enzyme may be added to the fermentation medium before the addition of the fermenting organism(s), such as yeast, but may also be added together with or after addition of the fermenting organism(s). It is preferred to add at least one surfactant and at least one carbohydrate-source generating enzyme before the initiation of the fermentation. However, it is also within the scope of the invention to add at least one surfactant and at least one carbohydrate-source generating enzyme during fermentation, such as after initiation of the fermentation.

At least one surfactant and at least one carbohydrate-source generating enzyme may be applied in an effective amount before and/or during fermentation. At least one surfactant and at least one carbohydrate-source generating enzyme may be applied in an effective amount before fermentation, such as, during propagation of the fermenting microorganism(s) or after propagation of the fermenting microorganism(s).

In an embodiment, the fermentation process of the present invention is carried out as a simultaneous saccharification and fermentation step (SSF) or a simultaneous liquefaction, saccharification and fermentation step (LSF). The fermentation according to the invention may be performed on un-gelatinized and/or un-liquefied raw starch (see, e.g., U.S. Pat. No. 4,316,956, WO 03/066816, WO 03/066826 and U.S. patent application No. 60/453,326 (WO 2004/080923) which are all hereby incorporated by reference). In addition to at least one surfactant and at least one carbohydrate-source generating enzyme other enzymatic activities may be added. Such enzyme activities include esterase activity, preferably lipase, phospholipase, and/or cutinase activity, laccase activity, phytase activity, cellulase activity, cellobiase activity, xylanase activity, or mixtures thereof.

In a preferred embodiment, the fermentation process is used for producing an alcohol, preferably ethanol. The presence of a surfactant and at least one carbohydrate-source generating enzyme may be used to raise the ethanol yield. In a preferred embodiment the fermentation is performed in the presence of one or more additional enzyme activities. The additional enzyme(s) may be introduced prior to, during/simultaneous with or after at least one surfactant and at least one carbohydrate-source generating enzyme. Additional enzyme activities contemplated are esterase, such as lipase, phospholipase and/or cutinase, phytase, laccase, protease, cellulase, cellobiase, or a mixture thereof.

### Growth Stimulators

In another embodiment of the present invention stimulator(s) for growth of the fermenting microorganism is(are) added/present in combination with at least one surfactant and at least one carbohydrate-source generating enzyme and optionally other enzyme activities. Preferred stimulators for growth, include vitamins and minerals.

### Additional Enzymes

In a preferred embodiment of the process of the invention one or more additional enzyme activities are used in combination with at least one surfactant and at least one carbohydrate-source generating enzyme. Preferred additional enzymes are esterases, such as lipases, phospholipases, and/or cutinases, phytase, laccase, proteases, cellulose, cellobiases, xylases, or mixtures thereof.

### Esterases

In a preferred embodiment of the invention at least one surfactant and at least one carbohydrate-source generating enzyme is applied in an effective amount prior to or during fermentation in combinations with an effective amount of esterase. The enzymes may be added prior to or during fermentation, including during or after the propagation of the fermenting microorganisms.

As used herein, an “esterase” also referred to as a carboxylic ester hydrolyases, refers to enzymes acting on ester bonds, and includes enzymes classified in EC 3.1.1 Carboxylic Ester Hydrolases according to Enzyme Nomenclature (available at http://www.chem.qmw.ac.uk/iubmb/enzyme or from Enzyme Nomenclature 1992, Academic Press, San Diego, Calif., with Supplement 1 (1993), Supplement 2 (1994), Supplement 3 (1995), Supplement 4 (1997) and Supplement 5, in Eur. J. Biochem. 1994, 223, 1-5; Eur. J. Biochem. 1995, 232, 1-6; Eur. J. Biochem. 1996, 237, 1-5; Eur. J. Biochem. 1997, 250; 1-6, and Eur. J. Biochem. 1999, 264, 610-650; respectively). Non-limiting examples of esterases include arylesterase, triacylglycerol lipase, acetylesterase, acetylcholinesterase, cholinesterase, tropinesterase, pectinesterase, sterol esterase, chlorophyllase, L-arabinonolactonase, gluconolactonase, uronolactonase, tannase, retinyl-palmitate esterase, hydroxybutyrate-dimer hydrolase, acylglycerol lipase, 3-oxoadipate enol-lactonase, 1,4-lactonase, galactolipase, 4-pyridoxolactonase, acylcamitine hydrolase, aminoacyl-tRNA hydrolase, D-arabinonolactonase, 6-phosphogluconolactonase, phospholipase A1, 6-acetylglucose deacetylase, lipoprotein lipase, dihydrocoumarin lipase, limonin-D-ring-lactonase, steroid-lactonase, triacetate-lactonase, actinomycin lactonase, orsellinate-depside hydrolase, cephalosporin-C deacetylase, chlorogenate hydrolase, alpha-amino-acid esterase, 4-methyloxaloacetate esterase, carboxymethylenebutenolidase, deoxylimonate A-ring-lactonase, 2-acetyl-1-alkylglycerophosphocholine esterase, fusarinine-C ornithinesterase, sinapine esterase, wax-ester hydrolase, phorbol-diester hydrolase, phosphatidylinositol deacylase, sialate O-acetylesterase, acetoxybutynylbithiophene deacetylase, acetylsalicylate deacetylase, methylumbelliferyl-acetate deacetylase, 2-pyrone-4,6-dicarboxylate lactonase, N-acetylgalactosaminoglycan deacetylase, juvenile-hormone esterase, bis(2-ethylhexyl)phthalate esterase, protein-glutamate methylesterase, 11-cis-retinyl-palmitate hydrolase, all-trans-retinyl-palmitate hydrolase, L-rhamnono-1,4-lactonase, 5-(3,4-diacetoxybut-1-ynyl)-2,2′-bithiophene deacetylase, fatty-acyl-ethyl-ester synthase, xylono-1,4-lactonase, N-acetylglucosaminylphosphatidylinositol deacetylase, cetraxate benzylesterase, acetylalkylglycerol acetylhydrolase, and acetylxylan esterase.

Preferred esterases for use in the present invention are lipolytic enzymes, such as, lipases (as classified by EC 3.1.1.3, EC 3.1.1.23 and/or EC 3.1.1.26) and phospholipases (as classified by EC 3.1.1.4 and/or EC 3.1.1.32, including lysophospholipases as classified by EC 3.1.1.5). Other preferred esterases are cutinases (as classified by EC 3.1.1.74).

The esterase may be added in an amount effective to obtain the desired benefit to improve the performance of the fermenting microorganism, e.g., to change the lipid composition/concentration inside and/or outside of the fermenting microorganism or in the cell membrane of the fermenting microorganism, to result in an improvement in the movement of solutes into and/or out of the fermenting microorganisms during fermentation and/or to provide more metabolizable energy sources (such as, e.g., by converting components, such as, oil from the corn substrate, to components useful the fermenting microorganism, e.g., unsaturated fatty acids and glycerol), to increase ethanol yield. Examples of effective amounts of esterase are from 0.01 to 400 LU/g DS (Dry Solids). Preferably, the esterase is used in an amount of 0.1 to 100 LU/g DS, more preferably 0.5 to 50 LU/g DS, and even more preferably 1 to 20 LU/g DS. Further optimization of the amount of esterase can hereafter be obtained using standard procedures known in the art.

In a preferred embodiment the esterase is a lipolytic enzyme, more preferably, a lipase. As used herein, “lipolytic enzymes” refers to lipases and phospholipases (including lyso-phospholipases). The lipolytic enzyme is preferably of microbial origin, in particular of bacterial, fungal or yeast origin. The lipolytic enzyme used may be derived from any source, including, for example, a strain of Absidia, in particular Absidia blakesleena and Absidia corymbifera, a strain of Achromobacter, in particular Achromobacter iophagus, a strain of Aeromonas, a strain of Alternaria, in particular Alternaria brassiciola, a strain of Aspergillus, in particular Aspergillus niger and Aspergillus flavus, a strain of Achromobacter, in particular Achromobacter iophagus, a strain of Aureobasidium, in particular Aureobasidium puliulans, a strain of Bacillus, in particular Bacillus pumilus, Bacillus strearothermophilus and Bacillus subtilis, a strain of Beauveria, a strain of Brochothtix, in particular Brochothrix thernosohata, a strain of Candida, in particular Candida cylindracea (Candida rugosa), Candida paralipolytica, and Candida antarctica, a strain of Chromobacter, in particular Chromobacter viscosum, a strain of Coprinus, in particular Coprinus cinerius, a strain of Fusarium, in particular Fusarium oxysporum, Fusarium solani, Fusarium solanipisi, and Fusarium roseum culmorum, a strain of Geotricum, in particular Geotricum penicillatum, a strain of Hansenula, in particular Hansenula anomala, a strain of Humicola, in particular Humicola brevispora, Humicola brevis var. thermoidea, and Humicola insolens, a strain of Hyphozyma, a strain of Lactobacillus, in particular Lactobacillus curvatus, a strain of Metarhizium, a strain of Mucor, a strain of Paecilomyces, a strain of Penicillium, in particular Penicillium cyclopium, Penicillium crustosum and Penicillium expansum, a strain of Pseudomonas in particular Pseudomonas aeruginosa, Pseudomonas alcaligenes, Pseudomonas cepacia (syn. Burkholdena cepacia), Pseudomonas fluorescens, Pseudomonas fragi, Pseudomonas maftophilia, Pseudomonas mendocina, Pseudomonas mephitica lipolytica, Pseudomonas alcaligenes, Pseudomonas plantari, Pseudomonas pseudoalcaligenes, Pseudomonas putida, Pseudomonas stutzeri, and Pseudomonas wisconsinensis, a strain of Rhizoctonia, in particular Rhizoctonia solani, a strain of Rhizomucor, in particular Rhizomucor miehei, a strain of Rhizopus, in particular Rhizopus japonicus, Rhizopus microsporus and Rhizopus nodosus, a strain of Rhodosporidium, in particular Rhodosporidium toruloides, a strain of Rhodotorula, in particular Rhodotorula glutinis, a strain of Sporobolomyces, in particular Sporobolomyces shibatanus, a strain of Thermomyces, in particular Thermomyces lanuginosus (formerly Humicola lanuginosa), a strain of Thiarosporella, in particular Thiarosporella phaseolina, a strain of Trichoderma, in particular Trichoderma harzianum, and Trichoderma reesei, and/or a strain of Verticillium.

In a preferred embodiment, the lipolytic enzyme is derived from a strain of Aspergillus, a strain of Achromobacter, a strain of Bacillus, a strain of Candida, a strain of Chromobacter, a strain of Fusarium, a strain of Humicola, a strain of Hyphozyma, a strain of Pseudomonas, a strain of Rhizomucor, a strain of Rhizopus, or a strain of Thermomyces.

In more preferred embodiments, the lipolytic enzyme is a lipase. Lipases may be applied herein for their ability to modify the structure and composition of triglyceride oils and fats in the fermentation media (including fermentation yeast), for example, resulting from a corn substrate. Lipases catalyze different types of triglyceride conversions, such as hydrolysis, esterification and transesterification. Suitable lipases include acidic, neutral and basic lipases, as are well-known in the art, although acidic lipases (such as, e.g., the lipase G AMANO 50, available from Amano) appear to be more effective at lower concentrations of lipase as compared to either neutral or basic lipases. Preferred lipases for use in the present invention included Candida antarcitca lipase and Candida cylindracea lipase. More preferred lipases are purified lipases such as Candida antarcitca lipase (lipase A), Candida antarcitca lipase (lipase B), Candida cylindracea lipase, and Penicillium camembertii lipase.

The lipase the one disclosed in EP 258,068-A or may be a lipase variant such as a variant disclosed in WO 00/60063 or WO 00/32758 which is hereby incorporated by reference. Preferred commercial lipases include LECITASE™, LIPOLASE™ and LIPEX™ (available from Novozymes A/S, Denmark) and G AMANO™ 50 (available from Amano).

Lipases are preferably added in amounts from about 1 to 400 LU/g DS, preferably 1 to 10 LU/g DS, and more preferably 1 to 5 LU/g DS.

In another preferred embodiment of the present invention, the at least one esterase is a cutinase. Cutinases are enzymes which are able to degrade cutin. The cutinase may be derived from any source. In a preferred embodiment, the cutinase is derived from a strain of Aspergillus, in particular Aspergillus oryzae, a strain of Alternaria, in particular Alternaria brassiciola, a strain of Fusarium, in particular Fusarium solani, Fusarium solani pisi, Fusarium roseum culmorum, or Fusarium roseum sambucium, a strain of Helminthosporum, in particular Helminthosporum sativum, a strain of Humicola, in particular Humicola insolens, a strain of Pseudomonas, in particular Pseudomonas mendocina, or Pseudomonas putida, a strain of Rhizoctonia, in particular Rhizoctonia solani, a strain of Streptomyces, in particular Streptomyces scabies, or a strain of Ulocladium, in particular Ulocladium consortiale. In a most preferred embodiment the cutinase is derived from a strain of Humicola insolens, in particular the strain Humicola insolens DSM 1800. Humicola insolens cutinase is described in WO 96/13580 which is hereby incorporated by reference. The cutinase may be a variant such as one of the variants disclosed in WO 00/34450 and WO 01/92502 which is hereby incorporated by reference. Preferred cutinase variants include variants listed in Example 2 of WO 01/92502 which are hereby specifically incorporated by reference. An effective amount of cutinase is between 0.01 and 400 LU/g DS, preferably from about 0.1 to 100 LU/g DS, more preferably, 1 to 50 LU/g DS. Further optimization of the amount of cutinase can hereafter be obtained using standard procedures known in the art.

In another preferred embodiment, the at least one esterase is a phospholipase. As used herein, the term phospholipase is an enzyme which has activity towards phospholipids. Phospholipids, such as lecithin or phosphatidylcholine, consist of glycerol esterified with two fatty acids in an outer. (sn-1) and the middle (sn-2) positions and esterified with phosphoric acid in the third position; the phosphoric acid, in turn, may be esterified to an aminoalcohol. Phospholipases are enzymes which participate in the hydrolysis of phospholipids. Several types of phospholipase activity can be distinguished, including phospholipases A₁ and A₂ which hydrolyze one fatty acyl group (in the sn-1 and sn-2 position, respectively) to form lysophospholipid; and lysophospholipase (or phospholipase B) which can hydrolyze the remaining fatty acyl group in lysophospholipid. Phospholipase C and phospholipase D (phosphodiesterases) release diacyl glycerol or phosphatidic acid, respectively.

The term phospholipase includes enzymes with phospholipase activity, e.g., phospholipase A (A₁ or A₂), phospholipase B activity, phospholipase C activity or phospholipase D activity. The term “phospholipase A” used herein in connection with an enzyme of the invention is intended to cover an enzyme with Phospholipase A₁ and/or Phospholipase A₂ activity. The phospholipase activity may be provided by enzymes having other activities as well, such as, e.g., a lipase with phospholipase activity. The phospholipase activity may, e.g., be from a lipase with phospholipase side activity. In other embodiments of the invention the phospholipase enzyme activity is provided by an enzyme having essentially only phospholipase activity and wherein the phospholipase enzyme activity is not a side activity.

The phospholipase may be of any origin, e.g., of animal origin (such as, e.g., mammalian), e.g. from pancreas (e.g. bovine or porcine pancreas), or snake venom or bee venom. Alternatively, the phospholipase may be of microbial origin, e.g. from filamentous fungi, yeast or bacteria, such as the genus or species Aspergillus, e.g., A. niger; Dictyostelium, e.g. D. discoideum; Mucor, e.g. M. javanicus, M. mucedo, M. subtilissimus; Neurospora, e.g. N. crassa; Rhizomucor, e.g., R. pusillus; Rhizopus, e.g. R. arrhizus, R. japonicus, R. stolonifer; Sclerotinia, e.g., S. libertiana; Trichophyton, e.g. T. rubrum; Whetzelinia, e.g. W. sclerotiorum; Bacillus, e.g., B. megaterium, B. subtilis; Citrobacter, e.g., C. freundii; Enterobacter, e.g., E. aerogenes, E. cloacae Edwardsiella, E. tarda; Erwinia, e.g., E. herbicola; Escherichia, e.g., E. coli; Klebsiella, e.g., K. pneumoniae; Proteus, e.g., P. vulgaris; Providencia, e.g. P. stuartii; Salmonella, e.g. S. typhimurium; Serratia, e.g., S. liquefasciens, S. marcescens; Shigella, e.g., S. flexneri; Streptomyces, e.g., S. violeceoruber, Yersinia, e.g., Y. enterocolitica. Thus, the phospholipase may be fungal, e.g., from the class Pyrenomycetes, such as the genus Fusarium, such as a strain of F. culmorum, F. heterosporum, F. solani, or a strain of F. oxysporum. The phospholipase may also be from a filamentous fungus strain within the genus Aspergillus, such as a strain of Aspergillus awamori, Aspergillus foetidus, Aspergillus japonicus, Aspergillus niger or Aspergillus oryzae. Preferred commercial phospholipases include LECITASE™ and LECITASE™ ULTRA (available from Novozymes A/S, Denmark).

An effective amount of phosphorlipase is between 0.01 and 400 LU/g DS, preferably from about 0.1 to 100 LU/g DS, more preferably, 1 to 50 LU/g DS. Further optimization of the amount of phospholipase can hereafter be obtained using standard procedures known in the art.

### Proteases

In another preferred embodiment of the process of the invention at least one surfactant and at least one carbohydrate-source generating enzyme is used in combination with at least one protease. The protease may be used, e.g., to digest protein to produce free amino nitrogen (FAN). Such free amino acids function as nutrients for the yeast, thereby enhancing the growth of the yeast and, consequently, the production of ethanol.

The fermenting microorganism for use in a fermentation process may be produced by propagating the fermenting microorganism in the presence of at least one protease. Although not limited to any one theory of operation, it is believed that the propagation of the fermenting microorganism with an effective amount of at least one protease reduces the lag time of the fermenting microorganism when the fermenting microorganism is subsequently used in a fermentation process as compared to a fermenting microorganism that was propagated under the same conditions without the addition of the protease. The action of the protease in the propagation process is believed to directly or indirectly result in the suppression or expression of genes which are detrimental or beneficial, respectively, to the fermenting microorganism during fermentation, thereby decreasing lag time and resulting in a faster fermentation cycle.

Proteases are well known in the art and refer to enzymes that catalyze the cleavage of peptide bonds. Suitable proteases include fungal and bacterial proteases. Preferred proteases are acidic proteases, i.e., proteases characterized by the ability to hydrolyze proteins under acidic conditions below pH 7. Suitable acid fungal proteases include fungal proteases derived from Aspergillus, Mucor, Rhizopus, Candida, Coiolus, Endothia, Enthomophtra, Irpex, Penicillium, Sclerotium and Torulopsis. Especially contemplated are proteases derived from Aspergillus niger (see, e.g., Koaze et al., (1964), Agr. Biol. Chem. Japan, 28, 216), Aspergillus saitoi (see, e.g., Yoshida, (1954) J. Agr. Chem. Soc. Japan, 28, 66), Aspergillus awamori (Hayashida et al., (1977) Agric. Biol. Chem., 42(5), 927-933, Aspergillus aculeatus (WO 95/02044), or Aspergillus oryzae; and acidic proteases from Mucor pusillus or Mucor miehei.

Bacterial proteases, which are not acidic proteases, include the commercially available products ALCALASE™ and NEUTRASE™ (available from Novozymes A/S). Other proteases include GC106 from Genencor Int. Inc., USA and NOVOZYM™ 50006 from Novozymes A/S, Denmark.

Preferably, the protease is an aspartic acid protease, as described, for example, Handbook of Proteolytic Enzymes, Edited by A. J. Barrett, N. D. Rawlings and J. F. Woessner, Academic Press, San Diego, 1998, Chapter 270). Suitable examples of aspartic acid protease include, e.g., those disclosed in R. M. Berka et al. Gene, 96, 313 (1990)); (R. M. Berka et al. Gene, 125, 195198 (1993)); and Gomi et al. Biosd. Biotech. Biochem. 57, 1095-1100 (1993), which are hereby incorporated by reference.

### Cellulase or Hemicellulases

Any suitable cellulase may be used according to the invention. The cellulase activity may be derived from any suitable origin; preferably, the cellulase is of microbial origin, such as derivable from a strain of a filamentous fungus (e.g., Aspergillus, Tnchoderna, Humicola, Fusarium). Preferably, the cellulase composition acts on both cellulosic and lignocellulosic material. Preferred cellulases for use in the present invention include exo-acting celluases and cellobiases, and combinations thereof. A preferably combination is an exo-acting cellulase and a cellobiase. Preferably, the cellulases have the ability to hydrolyze cellulose or lignocellulose under acidic conditions, i.e., below about pH 7. Examples of cellulases suitable for use in the present invention include, for example, CELLULCLAST™ (available from Novozymes A/S), NOVOZYM™ 188 (available from Novozymes A/S) Other commercially available preparations comprising cellulase which may be used include CELLUZYME™, CEREFLO™ and ULTRAFLO™ (Novozymes A/S), LAMINEX™ and SPEZYME™ CP (Genencor Int.) and ROHAMENT™ 7069 W (from Röhm GmbH). The cellulase enzymes are added in amounts effective of from about 0.001 to 0.5% wt. of solids, more preferably, 0.05% to 0.5% wt. of solids.

Preferred hemicellulase for use according to the present invention include xylanases, arabinofuranosidases, acetyl xylan esterase, glucuronidases, endo-galactanase, mannases, endo or exo arabinases, exo-galactanses, and mixtures thereof. Preferably, the hemicellulase for use in the present invention is an exo-acting hemicellulase, and more preferably, the hemicellulase is an exo-acting hemicellulase which has the ability to hydrolyze hemicellulose under acidic conditions of below pH 7. An example of hemicellulase suitable for use in the present invention includes VISCOZYME™ (available from Novozymes A/S, Denmark). The hemicellulase is added in an amount effective of from about 0.001 to 0.5% wt. of solids, more preferably, from about 0.05 to 0.5% wt. of solids.

### Xylanase

A xylanase (E.C. 3.2.1.8) activity may be derived from any suitable source, including fungal and bacterial organisms, such as Aspergillus, Disporotrichum, Penicillium, Neurospora, Fusarium and Trichoderma. Preferred commercially available preparations comprising xylanase include SHEARZYME®, BIOFEED WHEAT®, CELLUCLAST®, ULTRAFLO®, VISCOZYME® (Novozymes A/S) and SPEZYME® CP (Genencor Int.).

### Production of Enzymes

The enzymes referenced herein may be derived or obtained from any suitable origin, including, bacterial, fungal, yeast or mammalian origin. The term “derived” or means in this context that the enzyme may have been isolated from an organism where it is present natively, i.e., the identity of the amino acid sequence of the enzyme are identical to a native enzyme. The term “derived” also means that the enzymes may have been produced recombinantly in a host organism, the recombinant produced enzyme having either an identity identical to a native enzyme or having a modified amino acid sequence, e.g., having one or more amino acids which are deleted, inserted and/or substituted, i.e., a recombinantly produced enzyme which is a mutant and/or a fragment of a native amino add sequence or an enzyme produced by nucleic add shuffling processes known in the art. Within the meaning of a native enzyme are included natural variants. Furthermore, the term “derived” includes enzymes produced synthetically by, e.g., peptide synthesis. The term “derived” also encompasses enzymes which have been modified e.g. by glycosylation, phosphorylation, or by other chemical modification, whether in vivo or in vitro. The term “obtained” in this context means that the enzyme has an amino acid sequence identical to a native enzyme. The term encompasses an enzyme that has been isolated from an organism where it is present natively, or one in which it has been expressed recombinantly in the same type of organism or another, or enzymes produced synthetically by, e.g., peptide synthesis. With respect to recombinantly produced enzymes the terms “obtained” and “derived” refers to the identity of the enzyme and not the identity of the host organism in which it is produced recombinantly.

The enzymes may also be purified. The term “purified” as used herein covers enzymes free from other components from the organism from which it is derived. The term “purified” also covers enzymes free from components from the native organism from which it is obtained. The enzymes may be purified, with only minor amounts of other proteins being present. The expression “other proteins” relate in particular to other enzymes. The term “purified” as used herein also refers to removal of other components, particularly other proteins and most particularly other enzymes present in the cell of origin of the enzyme of the invention. The enzyme may be “substantially pure,” that is, free from other components from the organism in which it is produced, that is, for example, a host organism for recombinantly produced enzymes. In preferred embodiment, the enzymes are at least 75% (w/w) pure, more preferably at least 80%, at least 85%, at least 90%, at least 95%, at least 96%, at least 97%, at least 98%, or at least 99% pure. In another preferred embodiment, the enzyme is 100% pure.

The enzymes used in the present invention may be in any form suitable for use in the processes described herein, such as, e.g., in the form of a dry powder or granulate, a non-dusting granulate, a liquid, a stabilized liquid, or a protected enzyme. Granulates may be produced, e.g., as disclosed in U.S. Pat. Nos. 4,106,991 and 4,661,452, and may optionally be coated by process known in the art. Liquid enzyme preparations may, for instance, be stabilized by adding stabilizers such as a sugar, a sugar alcohol or another polyol, lactic acid or another organic acid according to established process. Protected enzymes may be prepared according to the process disclosed in EP 238,216.

### Fermentation Stimulators

In accordance with another preferred embodiment, a fermentation stimulator may be used in combination with any of the enzymatic processes described herein to further improve the fermentation process, and in particular, the performance of the fermenting microorganism, such as, rate enhancement and ethanol yield. A “fermentation stimulator” refers to stimulators for growth of the fermenting microorganisms, in particular, yeast. Preferred fermentation stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. See, e.g., Alfenore et al., Improving ethanol production and viability of Saccharomyces cerevisia by a vitamin feeding strategy during fed-batch process,” Springer-Verlag (2002), which is hereby incorporated by reference. Examples of minerals include minerals and mineral salts that can supply nutrients comprising P, K, Mg, S, Ca, Fe, Zn, Mn, and Cu.

### Fermentation Process

“Fermentation” or “fermentation process” refers to any fermentation process or any process comprising a fermentation step. A fermentation process of the invention includes, without limitation, fermentation processes used to produce fermentation products including alcohols (e.g., ethanol, methanol, butanol, 1,3-propanediol); organic acids (e.g., citric acid, acetic acid, itaconic acid, lactic acid, gluconic acid, gluconate, lactic acid, succinic acid, 2,5 diketo-D-gluconic acid); ketones (e.g., acetone); amino acids (e.g., glutamic acid); gases (e.g., H₂ and CO₂), and more complex compounds, including, for example, antibiotics (e.g., penicillin and tetracycline); enzymes; vitamins (e.g., riboflavin, B₁₂, beta-carotene); hormones, and other compounds. Fermentation processes also include fermentation processes used in the consumable alcohol industry (e.g., beer and wine), dairy industry (e.g., fermented dairy products), leather industry and tobacco industry. Preferred fermentation processes include alcohol fermentation processes, as are well known in the art. Preferred fermentation processes are anaerobic fermentation processes, as are well known in the art.

In a preferred embodiment, the fermentation process of the invention is used in combination with a liquefaction process and/or saccharification process, in which besides at least one surfactant and at least one carbohydrate-source generating enzyme additionally may include enzymatic activities, such as esterase, such as lipase, phosphorlipase, and/or cutinase, phytase, laccase, cellulase, cellobiase, xylanase, or mixtures thereof.

### Fermentation Media

“Fermentation media” or “fermentation medium” refers to the environment in which the fermentation is carried out and which includes the fermentation substrate, that is, the carbohydrate source that is metabolized by the fermenting microorganism(s). The fermentation media, including fermentation substrate and other raw materials used in the fermentation process of the invention may be processed, e.g., by milling, liquefaction and saccharification processes or other desired processes prior to or simultaneously with the fermentation process. Accordingly, the fermentation medium can refer to the medium before the fermenting microorganism(s) is(are) added, such as, the medium in or resulting from a liquefaction and/or saccharification process, as well as the media which comprises the fermenting microorganisms, such as, the media used in a simultaneous saccharification and fermentation process (SSF) or simultaneous liquefaction-saccharification-fermentation (LSF) process.

### Fermenting Organism

“Fermenting microorganism” refers to any microorganism suitable for use in a desired fermentation process. Suitable fermenting microorganisms according to the invention are able to ferment, i.e., convert, sugars, such as glucose or maltose, directly or indirectly into the desired fermentation product. Examples of fermenting microorganisms include fungal organisms, such as yeast. Preferred yeast includes strains of the Saccharomyces spp., and in particular, Saccharomyces cerevisiae. Commercially available yeast include, e.g., Red Star®/Lesaffre Ethanol Red (available from Red Star/Lesaffre, USA), FALI (available from Fleischmann's Yeast, a division of Burns Philp Food Inc., USA), SUPERSTART (available from Alitech), GERT STRAND (available from Gert Strand AB, Sweden) and FERMIOL (available from DSM Specialties).

### Fermentation Substrate

Any suitable substrate or raw material may be used in a fermentation process of the present invention. The substrate is generally selected based on the desired fermentation product and the process employed, as is well known in the art. Examples of substrates suitable for use in the processes of present invention, include starch-containing materials, such as tubers, roots, whole grains, corns, cobs, wheat, barley, rye, milo or cereals, sugar-containing raw materials, such as molasses, fruit materials, sugar, cane or sugar beet, potatoes, and cellulose-containing materials, such as wood or plant residues. Suitable substrates also include carbohydrate sources, in particular, low molecular sugars DP₁₋₃ that can be metabolized by the fermenting microorganism, and which may be supplied by direct addition to the fermentation media.

### Liquefaction or Saccharification

Any liquefaction or saccharification may be used in combination with the fermentation process of the present invention. According to the present invention the saccharification and liquefaction may be carried out simultaneously or separately with the fermentation process. In a preferred embodiment of the present invention, the liquefaction, saccharification and fermentation processes are carried out simultaneously (LSF).

“Liquefaction” is a process in which milled (whole) grain raw material is broken down (hydrolyzed) into maltodextrins (dextrins). Liquefaction is often carried out as a three-step hot slurry process. The slurry is heated to between 60-95° C., preferably 8-85° C., and the enzymes are added to initiate liquefaction (thinning). The slurry is then jet-cooked at a temperature between 95-140° C., preferably 105-125° C. to complete gelanitization of the slurry. Then the slurry is cooled to 60-95° C. and more enzyme(s) is(are) added to finalize hydrolysis (secondary liquefaction). The liquefaction process is usually carried out at pH 4.5-6.5, in particular at a pH between 5 and 6. Milled and liquefied whole grains are known as mash.

The liquefaction processes are typically carried out using any of the alpha-amylase listed above in the “Amylase” section.

“Saccharification” is a process in which the maltodextrin (such as, produced from the liquefaction process) is converted to low molecular sugars DP₁₋₃ (i.e., carbohydrate source) that can be metabolized by the fermenting organism, such as, yeast. Saccharification processes are well known in the art and are typically performed enzymatically using a glucoamylase. Alternatively or in addition, alpha-glucosidases or acid alpha-amylases may be used. A full saccharification process may last up to from about 24 to about 72 hours, and is often carried out at temperatures from about 30 to 65° C., and at a pH between 4 and 5, normally at about pH 4.5. However, it is often more preferred to do a pre-saccharification step, lasting for about 40 to 90 minutes, at temperature of between 30-65° C., typically about 60° C., followed by complete saccharification during fermentation in a simultaneous saccharification and fermentation process (SSF).

The most widely used process in ethanol production is the simultaneous saccharification and fermentation (SSF) process, in which there is no holding stage for the saccharification, meaning that fermenting organism, such as the yeast, and enzyme(s) is(are) added together. In SSF processes, it is common to introduce a pre-saccharification step at a temperature above 50° C., just prior to the fermentation.

More preferably, the liquefaction, saccharification or fermentation process is a simultaneous liquefaction-saccharification-fermentation (LSF) process or single enzymatic process, in which the liquefaction, saccharification and fermentation process are all carried out in one process, that is, all enzymes (or substitutable or additional non-enzyrnatic agents) used for liquefaction, saccharification and fermentation are added in the same process step, more preferably, simultaneously in the same process step. Preferred process conditions for LSF process include temperatures of about 26° C. to 40° C., preferably about 32° C., pH of about 4 to about 8, preferably about pH 5, and process times of about 48 to 72 hours, preferably about 72 hours.

Preferably, the LSF process or single enzymatic process is a raw starch hydrolysis (RSH) processes, more preferably, used in the production of alcohol, such as, e.g., ethanol. A “raw starch hydrolysis” process (RSH) differs from conventional starch treatment processes in that raw uncooked starch, also referred to as granular starch, is used in the ethanol fermentation process. As used herein, the term “granular starch” means raw uncooked starch, i.e. starch in its natural form found In cereal, tubers or grains. Starch is formed within plant cells as tiny granules insoluble in water. When put in cold water, the starch granules may absorb a small amount of the liquid and swell. At temperatures up to 50° C. to 75° C. the swelling may be reversible. However, with higher temperatures an irreversible swelling called gelatinization begins.

The term “initial gelatinization temperature” means the lowest temperature at which gelatinization of the starch commences. Starch heated in water begins to gelatinize between 50° C. and 75° C.; the exact temperature of gelatinization depends on the specific starch, and can readily be determined by the skilled artisan. Thus, the initial gelatinization temperature may vary according to the plant species, to the particular variety of the plant species as well as with the growth conditions. In the context of this invention the initial gelatinization temperature of a given starch is the temperature at which birefringence is lost in 5% of the starch granules using the process described by Gorinstein. S, and Lii. C., Starch/Stärke, Vol. 44 (12) pp. 461-466 (1992).

In accordance with a preferred embodiment at least one surfactant and at least one carbohydrate-source generating enzyme may be used, preferably in combination with an esterase, a phytase, laccase, cellulase, cellobiase, protease to increase ethanol yield in raw starch hydrolysis processes.

In a preferred embodiment, the present invention involves treating granular starch slurry with at least one surfactant and at least one carbohydrate-source generating enzyme and one or more of activity from the group of phytase, esterase, cellulase, cellobiase, protease, laccase, yeast at a temperature below the initial gelatinizatiion temperature of granular starch. A preferred amylase is an acid alpha-amylase, more preferably an acid fungal alpha-amylase.

In a more preferred embodiment, the raw starch hydrolysis process entails, treating granular starch slurry with a glucoamylase and/or alpha-amylase at a temperature between 0° C. and 20° C. below the initial gelatinization temperature of the granular starch, followed by treating the slurry with a glucoamylase and/or alpha amylase, yeast and at least one surfactant and at least one carbohydrate-source generating enzyme, and optionally an esterase, protease, phytase, laccase, cellulase, cellobiase, hemicellulase at a temperature of between 10° C. and 35-C.

In yet another preferred embodiment, the process entails the sequential steps of: (a) treating a granular starch slurry with an add alpha-amylase and a glucoamylase at a temperature of 0° C. to 20° C. below the initial gelatinization temperature of the granular starch, preferably for a period of 5 minutes to 12 hours, (b) treating the slurry in the presence of an acid alpha-amylase, a glucoamylase, a yeast and at least one surfactant and at least one carbohydrate-source generating enzyme, and optionally a phytase, protease, laccase, esterase cellulase, cellobiase, hemicellulase at a temperature of between 10° C. and 35° C., preferably for a period of 20 to 250 hours to produce ethanol.

In another preferred embodiment, a maltogenic alpha-amylase is used in combination with at least one surfactant and at least one carbohydrate-source generating enzyme in the RSH process.

A preferred application of the fermentation processes and compositions described herein is in an alcohol production process (such as, e.g., ethanol for use as a fuel or fuel additive), more preferably using a raw starch hydrolysis process.

Ethanol production processes generally involve the steps of milling, liquefaction, saccharification, fermentation and distillation. In the production of ethanol and other starch-based products, the raw material, such as whole grain, preferably corm, is milled in order to open up the structure and allow for further processing. Two processes are preferred according to the invention: wet milling and dry milling. Preferred for ethanol production is dry milling where the whole kernel is milled and used in the remaining part of the process. Wet milling may also be used and gives a good separation of germ and meal (starch granules and protein) and is with a few exceptions applied at locations where there is a parallel production of syrups. Both wet and dry milling processes are well known in the art.

In ethanol production, the fermenting organism is preferably yeast, which is applied to the mash. Preferred yeast is derived from Saccharomyces spp., more preferably, from Saccharomyces cerevisiae. In preferred embodiments, yeast is applied to the mash and the fermentation is ongoing for 24-96 hours, such as typically 35-60 hours. In preferred embodiments, the temperature is generally between 26-34° C., in particular about 32° C., and the pH is generally from pH 3-6, preferably around pH 4-5. Yeast cells are preferably applied in amounts of 10⁵ to 10¹², preferably from 10⁷ to 10¹⁰, especially 5×10⁷ viable yeast count per ml of fermentation broth. During the ethanol producing phase the yeast cell count should preferably be in the range from 10⁷ to 10¹⁰, especially around 2×10⁸. Further guidance in respect of using yeast for fermentation can be found in, e.g., “The alcohol Textbook” (Editors K. Jacques, T. P. Lyons and D. R. Kelsall, Nottingham University Press, United Kingdom 1999), which is hereby incorporated by reference.

Following fermentation, the mash may be distilled to extract the alcohol product (ethanol). In the case where the end product is ethanol, obtained according to the processes of the invention, it may be used as, e.g., fuel ethanol; drinking ethanol, i.e., potable neutral spirits; or industrial ethanol.

In an aspect the invention relates to a process for producing ethanol, comprising

(a) milling whole grains;

(b) liquefying the product of step (a);

(c) saccharifying the liquefied material;

(d) fermenting the saccharified material using a microorganism, wherein the fermentation process further comprises contacting the fermentation media with at least one surfactant, at least one carbohydrate generating enzyme.

In a preferred embodiment of the invention the carbohydrate-source generating enzyme is a glucoamylase or an alpha-amylase of mixtures thereof. Preferred alpha-amylases are acid fungal alpha-amylase. Preferred mixtures has a ratio of acid alpha-amylase activity (AFAU) per glucoamylase activity (AGU) (AFAU per AGU) of at least 0.1, in particular at least 0.16, such as in the range from 0.12 to 0.50.

The fermented product from step (d) may further be distilled using any method known in the art. In a preferred embodiment the process is a simultaneous liquefaction and saccharification process. (SSF), a simultaneous liquefaction, sacchariifcation and fermentation process (LSF). Preferably the LSF process is a raw starch hydrolysis (RSH) process. Additional enzymes used during the ethanol production process, especially in the fermentation step, include esterase, such as lipase, phospholipases and/or cutinase, phytase, cellulase, cellubiohydrolase, hemicellulase, and xylanase, or mixtures thereof.

The surfactant may be any of the above mentioned. Preferred is alcohol ethoxylates.

The fermenting microorganism may be yeast, preferably a yeast derived from Saccharomyces spp., more preferably, from Saccharomyces cerevisiae.

### Materials and Methods

### Enzymes:

Trichoderma reesei cellulase preparation: CELLUCLAST™ (available from Novozymes A/S, Denmark)
A. niger glucoamylase: SPIRIZYME FUEL (available from Novozymes A/S)

### Yeast:

Ethanol Red available from Red Star/Lesaffre, USA

### Surfactants:

TRITON® X100 from Union Carbide
BEROL® 087 from Akzo Nobel,
SOFTANOL® 90 from Honeywell & Stein

### Methods:

### Aloha-Amylase Activity (KNU)

The amylolytic activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e. at 37° C.+/−0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S, Denmark, which folder is hereby included by reference.

### Phytase Activity

The phytase activity is measured in FYT units, one FYT being the amount of enzyme that liberates 1 micromole inorganic ortho-phosphate per min. under the following conditions: pH 5.5; temperature 37° C.; substrate: sodium phytate (C₆H₆O₂₄P₆Na₁₂) at a concentration of 0.0050 mole/l.

### Determination of FAU Activity

One Fungal Alpha-Amylase Unit (FAU) is defined as the amount of enzyme, which breaks down 5.26 g starch (Merck Amylum solubile Erg. B.6, Batch 9947275) per hour based upon the following standard conditions:

| | |
|---|---|
| Substrate | Soluble starch |
| Temperature | 37° C. |
| PH | 4.7 |
| Reaction time | 7-20 minutes |

### Determination of Acid Aloha-Amylase Activity (AFAU)

Acid alpha-amylase activity is measured in AFAU (Acid Fungal Alpha-amylase Units), which are determined relative to an enzyme standard.

The standard used is AMG 300 L (from Novozymes A/S, Denmark, glucoamylase wild-type Aspergillus niger G1, also disclosed in Boel et al. (1984), EMBO J. 3 (5), p. 1097-1102) and WO 92/00381). The neutral alpha-amylase in this AMG falls after storage at room temperature for 3 weeks from approx. 1 FAU/mL to below 0.05 FAU/mL.

The acid alpha-amylase activity in this AMG standard is determined in accordance with the following description. In this method, 1 AFAU is defined as the amount of enzyme, which degrades 5.260 mg starch dry matter per hour under standard conditions.

Iodine forms a blue complex with starch but not with its degradation products. The intensity of colour is therefore directly proportional to the concentration of starch. Amylase activity is determined using reverse colorimetry as a reduction in the concentration of starch under specified analytic conditions.

| | | |
|---|---|---|
| | Alpha-amylase | |
| Starch + Iodine | → | Dextrins + Oligosaccharides |
| | 40° C., pH 2.5 | |
| Blue/violet | t = 23 sec. | Decoloration |

Standard conditions/reaction conditions: (per minute)

| | |
|---|---|
| Substrate: | Starch, approx. 0.17 g/L |
| Buffer: | Citate, approx. 0.03 M |
| Iodine (I₂): | 0.03 g/L |
| CaCl₂: | 1.85 mM |
| pH: | 2.50 ± 0.05 |
| Incubation temperature: | 40° C. |
| Reaction time: | 23 seconds |
| Wavelength: | lambda = 590 nm |
| Enzyme concentration: | 0.025 AFAU/mL |
| Enzyme working range: | 0.01-0.04 AFAU/mL |

If further details are preferred these can be found in EB-SM-0259.02101 available on request from Novozymes A/S, Denmark, and incorporated by reference.

### Acid Alpha-Amylase Units (MU)

The acid alpha-amylase activity can be measured in MU (Acid Alpha-amylase Units), which is an absolute method. One Acid Amylase Unit (MU) is the quantity of enzyme converting 1 g of starch (100% of dry matter) per hour under standardized conditions into a product having a transmission at 620 nm after reaction with an iodine solution of known strength equal to the one of a color reference.

Standard conditions/reaction conditions:

| | |
|---|---|
| Substrate: | Soluble starch. Concentration approx. |
| | 20 g DS/L. |
| Buffer: | Citrate, approx. 0.13 M, pH = 4.2 |
| Iodine solution: | 40.176 g potassium iodide + 0.088 g |
| | iodine/L |
| City water | 15°-20° dH (German degree hardness) |
| pH: | 4.2 |
| Incubation temperature: | 30° C. |
| Reaction time: | 11 minutes |
| Wavelength: | 620 nm |
| Enzyme concentration: | 0.13-0.19 AAU/mL |
| Enzyme working range: | 0.13-0.19 AAU/mL |

The starch should be Lintner starch, which is a thin-boiling starch used in the laboratory as colorimetric indicator. Lintner starch is obtained by dilute hydrochloric acid treatment of native starch so that it retains the ability to color blue with iodine. Further details can be found in EP Patent No. 140410, which disclosure is hereby incorporated by reference.

### Glucoamylase Activity (AGI)

Glucoamylase (equivalent to amyloglucosidase) converts starch into glucose. The amount of glucose is determined here by the glucose oxidase method for the activity determination. The method described in the section 76-11 Starch-Glucoamylase Method with Subsequent Measurement of Glucose with Glucose Oxidase in “Approved methods of the American Association of Cereal Chemists”. Vol. 1-2 AACC, from American Association of Cereal Chemists, (2000); ISBN: 1-891127-12-8.

One glucoamylase unit (AGI) is the quantity of enzyme which will form 1 micromol of glucose per minute under the standard conditions of the method.

Standard conditions/reaction conditions:

| | |
|---|---|
| Substrate: | Soluble starch. |
| | Concentration approx. 16 g dry matter/L. |
| Buffer | Acetate, approx. 0.04 M, pH = 4.3 |
| pH: | 4.3 |
| Incubation temperature: | 60° C. |
| Reaction time: | 15 minutes |
| Termination of | NaOH to a concentration of approximately |
| the reaction: | 0.2 g/L (pH ~9) |
| Enzyme concentration: | 0.15-0.55 AAU/mL. |

The starch should be Lintner starch, which is a thin-boiling starch used in the laboratory as colorimetric indicator. Lintner starch is obtained by dilute hydrochloric acid treatment of native starch so that it retains the ability to color blue with iodine.

### Glucoamylase Activity (AGU)

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

| AMG incubation: | |
|---|---|
| Substrate: | maltose 23.2 mM |
| Buffer: | acetate 0.1 M |
| pH: | 4.30 ± 0.05 |
| Incubation temperature: | 37° C. ± 1 |
| Reaction time: | 5 minutes |
| Enzyme working range: | 0.5-4.0 AGU/mL |

| Color reaction: | |
|---|---|
| GlucDH: | 430 U/L |
| Mutarotase: | 9 U/L |
| NAD: | 0.21 mM |
| Buffer: | phosphate 0.12 M; 0.15 M NaCl |
| pH: | 7.60 ± 0.05 |
| Incubation temperature: | 37° C. ± 1 |
| Reaction time: | 5 minutes |
| Wavelength: | 340 nm |

A folder (EB-SM-0131.02/01) describing this analytical method in more detail is available on request from Novozymes A/S, Denmark, which folder is hereby included by reference.

### Cutinase Activity (LU)

The cutinase activity is determined as lipolytic activity determined using tributyrine as substrate. This method was based on the hydrolysis of tributyrin by the enzyme, and the alkali consumption is registered as a function of time. One Lipase Unit (LU) is defined as the amount of enzyme which, under standard conditions (i.e. at 30° C.; pH 7.0; with Gum Arabic as emulsifier and tributyrine as substrate) liberates 1 micro mol titrable butyric add per minute. A folder AF 95/5 describing this analytical method in more detail is available upon request from Novozymes A/S, Denmark, which folder is hereby incorporated by reference.

### Xylanolytic Activity (FXU)

The xylanolytic activity can be expressed in FXU-units, determined at pH 6.0 with remazol-xylan (4-O-methyl-D-glucurono-D-xylan dyed with Remazol Brilliant Blue R, Fluka) as substrate.

A xylanase sample is incubated with the remazol-xylan substrate. The background of non-degraded dyed substrate is precipitated by ethanol. The remaining blue color in the supernatant (as determined spectrophotometrically at 585 nm) is proportional to the xylanase activity, and the xylanase units are then determined relatively to an enzyme standard at standard reaction conditions, i.e. at 50° C., pH 6.0, and 30 minutes reaction time.

A folder EB-SM-352.02/01 describing this analytical method in more detail is available upon request to Novozymes A/S, Denmark, which folder is hereby incorporated by reference.

### Cellulytic Activity (EGU)

The cellulytic activity may be measured in endo-glucanase units (EGU), determined at pH 6.0 with carboxymethyl cellulose (CMC) as substrate. A substrate solution is prepared, containing 34.0 g/l CMC (Hercules 7 LFD) in 0.1 M phosphate buffer at pH 6.0. The enzyme sample to be analyzed is dissolved in the same buffer. 5 ml substrate solution and 0.15 ml enzyme solution are mixed and transferred to a vibration viscosimeter (e.g. MIVI 3000 from Sofraser, France), thermostated at 40° C. for 30 minutes. One EGU is defined as the amount of enzyme that reduces the viscosity to one half under these conditions. The amount of enzyme sample should be adjusted to provide 0.01-0.02 EGU/ml in the reaction mixture. The arch standard is defined as 880 EGU/g.

A folder EB-SM-0275.02/01 describing this analytical method in more detail is available upon request to Novozymes A/S, Denmark, which folder is hereby incorporated by reference.

### Determination of Cellobiase Activity (CBU)

Cellobiase (beta-glucosidase EC 3.2.1.21) hydrolyzes beta-1,4 bonds in cellobiose to release two glucose molecules. The amount of glucose released is determined specifically and quantitatively using the hexokinase method as follows:

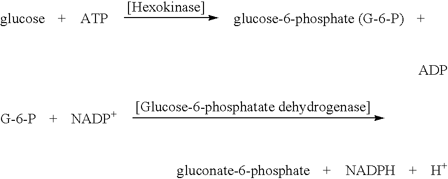

The increase in absorbance is then measured at 340 nm as the absorbance value for NADPH is high at this wavelength.

### Reaction Conditions

Reaction:

| Temperature: | 40° C. |
|---|---|
| pH: | 5.0 |

Detection:

| | |
|---|---|
| Reaction time: | 15 minutes |
| Wavelength: | 340 nm |

One cellobiase unit (CBU) is the amount of enzyme, which releases 2 micro moles glucose per minute under the standard conditions above with cellobiose as substrate.

A folder (EB-SM-0175.02102) describing this analytical method in more detail is available on request from Novozymes A/S, Denmark, which folder is hereby incorporated by reference.

### EXAMPLES

### Example 1

### Enhanced Production of Ethanol from Corn Using SOFTANOL® 90 Surfactants

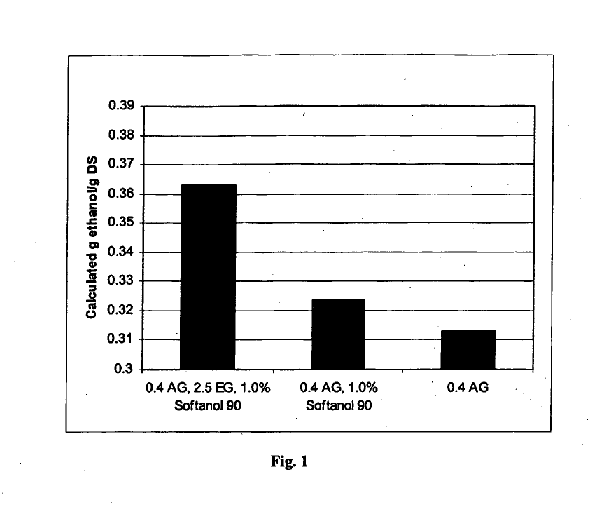

Ethanol Red yeast was propagated aerobically in corn mash which was ground for 5 minutes in a blender, then screened through #45 mesh to remove particulates. One gram of Urea (FISHER, US) and 200 mLs of tap water were added to every 300 grams of mash. 3 mg/L penicillin (SIGMA, US) was also added and the mash was then adjusted to pH 6 using sulfuric acid. A sample (250 g) of the prepared mash was weighed out, 32 microL A. niger glucoamylase (SPIRIZYME® FUEL) and the 0.12 g yeast were added, then the mixture was incubated at 32° C. with constant stirring (300 rpm) for approximately 24 hours. For the fermentation, approximately 5 grams of corn mash was introduced into 16 mL culture tubes. The cap on the culture tube was pierced with a needle to allow for gas release. A dry solids content was measured (31% w/w DS/g) and used for calculating the enzyme dose. 5 grams of mash was weighed out and 0.4 AGU/g DS of a glucoamylase (SPIRIZYME® FUEL), 2.5 EGU/g DS cellulase (CELLUCLAST®), and 1.0%/g DS of surfactant (SOFTANOL® 90) was added. The fermentation was carried out at 32° C. for about 65 hours. The ethanol percentage was determined by HPLC and compared to corresponding control fermentations comprising of only glucoamylase, and glucoamylase and surfactant. The result of the test is shown in FIG. 1.

### Example 2

### Enhanced Production of Ethanol from Corn Using Triton® X100 Surfactants

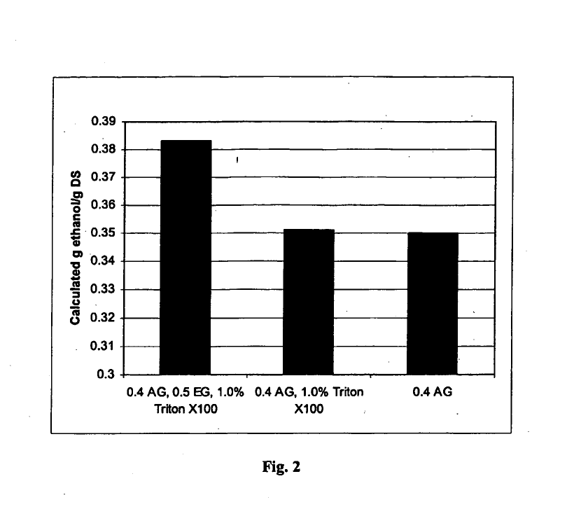

The test in Example 1 was repeated, except that 1.0% TRITON® X100 and 0.5 EGU/g DS cellulase (CELLUCLAST®) were used. The result of the test is shown in FIG. 2.

### Example 3

### Enhanced production of ethanol from corn using BEROL® 087 surfactants

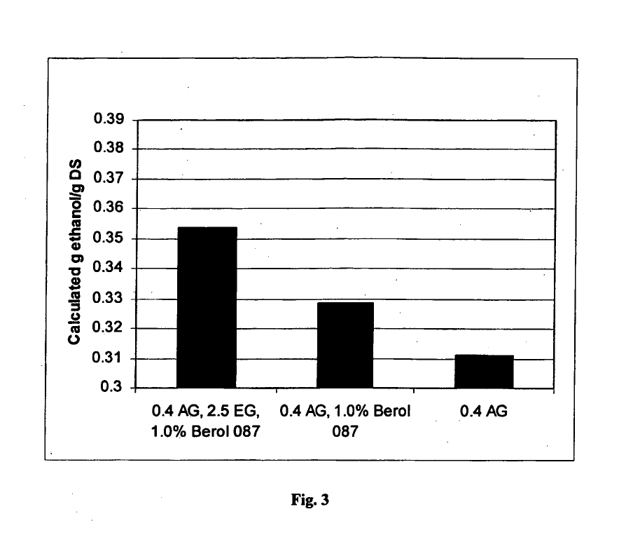

The test in Example 1 was repeated, except that 1.0% BEROL 087 and 2.5 EGU/g DS cellulase (CELLUCLAST®) were used. The result of the test is shown in FIG. 3.

1-20. (canceled)

21. A process for producing a fermentation product in a fermentation medium which process include a fermentation step, comprising subjecting the fermentation medium to at least one surfactant and at least one carbohydrate-source generating enzyme.

22. The process of claim 21, wherein the surfactant is an alcohol ethoxylate.

23. The process of claim 21, wherein fermentation is carried out using a yeast.

24. The process of claim 21, wherein said fermentation product is ethanol.

25. The process of claim 21, wherein the carbohydrate-source generating enzyme is glucoamylase or an alpha-amylase, or mixtures thereof.

26. The process of claim 21, wherein the carbohydrate-source generating enzyme is a mixture of acidic fungal alpha-amylase activity (AFAU) and glucoamylase activity (AGU) had having AFAU per AGU of at least 0.1.

27. The process of claim 21, wherein the carbohydrate-source generating enzyme is a mixture of acidic fungal alpha-amylase activity (AFAU) and glucoamylase activity (AGU) had having AFAU per AGU of at least 0.16.

28. The process of claim 25, wherein the glucoamylase is derived from a strain of the genus Corticium, Talaromyces, or Aspergillus.

29. The process of claim 21, wherein further a cellulase, cellobiase, or hemicellulase is present.

30. The process of claim 21, wherein fermentation step which is part of a simultaneous saccharification and fermentation process (SSF) or a liquefaction, saccharification, and fermentation process (LSF).

31. The process of claim 21, wherein the fermentation step is carried out in the presence of further one or more enzymes selected from the group consisting of an esterase, phytase, xylanase, laccase, protease, alpha-amylase, and glucoamylase.

32. The process of claim 21, wherein the fermentation is part of a dry milling process or of a wet milling process.

33. The process of claim 31, wherein the raw material for milling process is a starch-containing raw material, such as corn, wheat, barley, or milo.

34. The process of claim 21 wherein the glucoamylase is derived from a strain of C. rolfsii, T. or Aspergillus niger.

35. A process for producing ethanol, comprising
(a) milling whole grains;
(b) liquefying the product of step (a);
(c) saccharifying the liquefied material obtained in step (b);
(d) fermenting the saccharified material using a fermenting microorganism, wherein the fermentation process further comprises contacting the fermentation media with at least one surfactant, at least one carbohydrate-source generating enzyme.

36. The process of claim 35, wherein the surfactant is an alcohol ethoxylate.

37. The process of claim 35, wherein fermentation is carried out using a yeast.

38. The process of claim 35, wherein said fermentation product is ethanol.

39. The process of claim 35, wherein the carbohydrate-source generating enzyme is glucoamylase or an alpha-amylase, or mixtures thereof.

40. The process of claim 35, wherein the carbohydrate-source generating enzyme is a mixture of acidic fungal alpha-amylase activity (AFAU) and glucoamylase activity (AGU) had having AFAU per AGU of at least 0.1.
